(19) United States
(12) Patent Application Publication
(10) Pub. No.: US 2023/0099249 A1
(43) Pub. Date: Mar. 30, 2023

(54) AUTOMATED DATA-BASED PROVISION OF A PATIENT-SPECIFIC MEDICAL ACTION RECOMMENDATION

(71) Applicant: Siemens Healthcare GmbH, Erlangen (DE)

(72) Inventors: Christoph SPEIER, Erlangen (DE); Sven KOHLE, Erlangen (DE); Martin HUBER, Uttenreuth (DE)

(73) Assignee: Siemens Healthcare GmbH, Erlangen (DE)

(21) Appl. No.: 17/953,532

(22) Filed: Sep. 27, 2022

(30) Foreign Application Priority Data
Sep. 29, 2021 (DE) 10 2021 210 899.7

(51) Int. Cl. G16H 50/20; G16H 10/60

(52) U.S. Cl. CPC G16H 50/20; G16H 10/60; G16H 30/20

(57) ABSTRACT

In a computer-implemented method for providing a patient-specific medical action recommendation, a data record of the patient is received, and based on the data record, one or more observables are automatically determined. The one or more observables describe a clinical condition of the patient. The one or more observables are displayed via a user interface, and user input regarding the displayed one or more observables is received via the user interface. Based on the user input and, optionally, the data record, an action recommendation is provided to the user regarding further steps for diagnosing/treating the patient. An action recommendation device and a clinical examination and treatment system are also described.

### CROSS-REFERENCE TO RELATED APPLICATION(S)

The present application claims priority under 35 U.S.C. § 119 to German Patent Application No. 10 2021 210 899.7, filed Sep. 29, 2021, the entire contents of which are incorporated herein by reference.

### FIELD

One or more example embodiments of the present invention relate to a method for providing a patient-specific medical action recommendation. One or more example embodiments of the present invention also relate to an action recommendation device. One or more example embodiments of the present invention further relate to a clinical examination and treatment system.

### BACKGROUND

Nowadays, the operation of hospitals is subject to very high rationalization pressure and exacting demands on the effectiveness of the processes taking place there. There is often little time for medical staff to familiarize themselves with a patient's case and to make decisions for the best possible examination as a prerequisite for correct assessment or diagnosis and subsequent effective and appropriate treatment of the patient.

With the aid of modern imaging techniques, two- or three-dimensional image data is often generated which can be used for visualization of an imaged object under examination and also for other applications. The generated image data of a patient is stored in a database. Such a database is usually part of a PACS central image management system (PACS=Picture Archiving and Communication System) for all radiological images. Additional patient data can be retrieved via a radiology information system RIS in which the administrative data of all actions relating to a patient are stored. If an attending physician now receives the aforementioned data about a patient, they are on their own as regards taking further action based on the data received. Often, the attending physician also lacks additional data for making a correct decision for further action.

### SUMMARY

An important aspect of such a workflow for examining a patient is also the preparation, setup and supplying with suitable data of the medical equipment used, especially medical imaging devices, in order to obtain an image result that can be used for subsequent diagnosis. In this context, automated adjustment and control of the technical systems used would be desirable in order to reduce the workload of medical staff and eliminate or reduce errors in the preparation of the examination processes using technical systems.

The inventors have identified a problem of organizing an examination and treatment process of a patient more effectively than has hitherto been the case in the daily routine of a hospital.

This object is achieved by a method for providing a patient-specific medical action recommendation, an action recommendation device, and a clinical examination and treatment system, according to one or more example embodiments of the present invention.

In the inventive, preferably computer-implemented method for providing a patient-specific medical action recommendation, a data record, preferably comprising a medical record, of a patient is received, preferably from a database. Such a data record of a patient can, for example, comprise medical data from the PACS of a hospital and/or administrative data from the RIS concerning the patient. Based on the data record, one or more observables are determined, preferably automatically. The one or more observables describe a clinical condition of the patient.

The observables include condition attributes or symptoms of the patient. Condition attributes pertain to information about a patient's state of health. Such information can, for example, relate to whether or not the patient is a smoker or whether or not the patient's condition is acute. Condition attributes can also include presumptive or probabilistic information about a patient's condition, for example information that metastases are suspected or not suspected, or laboratory values are good or bad, i.e. suggest a better or worse state of health. Such information may also include examination results, such as a biopsy result, and indicate whether such a biopsy result is available at all. A condition attribute can also extend to whether or not particular treatment steps were performed, for example whether or not a particular medication was administered. In addition, the observable can include pre-existing diagnoses, for example a plurality of differential diagnoses. Such a differential diagnosis can, for example, relate to the distinction between the occurrence of an interstitial lung disease and a lung carcinoma.

In addition, as part of the method according to one or more example embodiments of the present invention, the one or more observables are displayed via a user interface to a user, i.e. a health care professional, for example a patient's attending physician.

A user input from a user relating to the displayed observables is then received via the user interface and an action recommendation for the user regarding further steps for diagnosing and/or treating the patient is output based on the user input and, optionally, the patient's data record.

The user input from the user, for example an attending physician, can include confirming or rejecting an observable, evaluating an observable, or other user responses to observables displayed to them. For example, a user can also change individual observables based on individual information and knowledge about their patient.

The action recommendation contains advice for medical action for the user. For example, the action recommendation can be related to carrying out examinations and, in particular, may suggest further examinations. In this context, a biopsy or laboratory data may be requested, for example. In particular, this action recommendation can also be made “on the fly”, i.e. directly during a medical examination or treatment process. For example, even while image acquisition of a patient is in progress, a different contrast agent can be suggested or image acquisition parameters can be changed during the image acquisition process.

The action recommendation can also be related to the assessment of the patient's existing data record and give the user advice as to what data to look at and what to check in the data. For example, the action recommendation may include advice to check if there are metastases in a particular organ, or to look at a particular image dataset for that purpose, or advice as to what analysis tools can be appropriately applied to that particular image dataset.

The action recommendation can include a suggestion for upcoming treatment steps, such as the use of a particular drug or radiation.

The action recommendation can also include a recommendation to involve further experts or to refer the patient.

The action recommendation can be regarded as a suggestion. However, if the user confirms this suggestion, the action recommendation can also be implemented directly in the sense of controlling a technical system, for example by triggering an imaging modality or booking a biopsy appointment.

Advantageously, a user of the method according to one or more example embodiments of the present invention does not have to perform any active steps to plan a workflow for examining and possibly also for treating a patient. Rather, the steps of inspecting the patient data and evaluating it, as well as reacting thereto in the form of planning a workflow, are performed automatically. However, the user is still fully involved in inspecting the patient data and in planning the workflow, so that they retain full control over these processes and can intervene at any intermediate stage of the planning. To this end, the observables determined during data collection and evaluation are advantageously displayed to the user and can be assessed, confirmed or rejected, or supplemented by the user. In addition, the user can also react to the action recommendation by changing, confirming or rejecting said action recommendation. The user's effort and workload requirements are thus reduced by the automated action recommendation without the user having to relinquish control over the process of planning a workflow in relation to a patient. Consequently, clinical processes can be made more efficient without reducing patient safety, for example due to anomalies occurring during the automated evaluation of patient data.

The action recommendation device according to one or more example embodiments of the present invention has a data receiving interface for receiving a preferably medical data record of a patient. Also a component part of the action recommendation device according to one or more example embodiments of the present invention is a determination unit for automatically determining one or more observables based on the data record, wherein the one or more observables describe the patient's clinical condition. The action recommendation device according to one or more example embodiments of the present invention comprises a user interface for displaying the one or more observables and for receiving a user input from a user relating to the displayed observables. The action recommendation device according to one or more example embodiments of the present invention further comprises a recommendation providing unit for providing an action recommendation to the user regarding further steps for diagnosing and/or treating the patient based on the user input and, optionally, the patient's data record. The action recommendation device according to one or more example embodiments of the present invention shares the advantages of the preferably computer-implemented method for providing a patient-specific medical action recommendation.

The clinical examination and treatment system according to one or more example embodiments of the present invention incorporates the action recommendation device according to one or more example embodiments of the present invention. The clinical examination and treatment system further comprises one or more examination devices, such as for example a medical imaging device or a treatment device. The examination and/or treatment device can be controlled based on an action recommendation of the action recommendation device. That is to say, the action recommendation can contain instructions or protocol data which, after confirmation by the user, are transmitted to the examination and/or treatment device to carry out a planned examination or treatment procedure as the case may be. The clinical examination and treatment system shares the advantages of the method according to one or more example embodiments of the present invention for providing a patient-specific medical action recommendation.

As already indicated, the essential components of the action recommendation device according to one or more example embodiments of the present invention can be predominantly in the form of software components. This relates in particular to the determination unit and the recommendation providing unit. In principle, however, these components can also be implemented in some cases, especially when particularly fast calculations are involved, in the form of software-supported hardware, for example FPGAs or the like, or by using processors. Likewise, the required interfaces can be designed as software interfaces, for example when it is only a matter of transferring data from other software components. However, they can also be designed as hardware interfaces that are controlled by suitable software.

The advantage of a largely software-based implementation is that computer units already in use in clinical examination and treatment systems can be easily upgraded by a software update in order to operate in the manner according to one or more example embodiments of the present invention. In this respect, the object is also achieved by a corresponding computer program product comprising a computer program which can be loaded directly into a memory device of a computer unit of a clinical examination and treatment system and comprises program sections for carrying out all the steps of the method according to one or more example embodiments of the present invention when the computer program is executed in the computer unit of the clinical examination and treatment system.

As well as the computer program, such a computer program product may comprise additional elements such as documentation and/or additional components, including hardware components, such as hardware keys (dongles, etc.) for using the software.

For transfer to the storage device of a computer unit of the clinical examination and treatment system and/or storage on the computer unit of the clinical examination and treatment system, a computer-readable medium, for example a memory stick, a hard disk or another transportable or permanently installed data carrier can be used on which are stored the program sections of the computer program that can be read in and executed by a computer unit. For this purpose, the computer unit can have, for example, one or more cooperating microprocessors or the like.

The dependent claims and the following description each contain particularly advantageous embodiments and further developments of one or more example embodiments of the present invention. In particular, the claims of one claim category can also be further developed analogously to the dependent claims of another claim category. Moreover, the various features of different exemplary embodiments and claims can also be combined to form new exemplary embodiments within the scope of the present invention.

In the method according to one or more example embodiments of the present invention for providing a patient-specific medical action recommendation, determining the one or more observables preferably involves applying a state analysis algorithm to the medical data record. The state analysis algorithm is designed to extract observables from medical data records.

The state analysis algorithm can, for example, be AI-based (AI=artificial intelligence). Such an AI-based structure can preferably comprise a neural network that extracts features/observables directly from patient data, or a classification algorithm, such as “k-nearest neighbors”, that sorts a patient into event space and assigns observables depending on the location in event space. The application of such a classification algorithm is described, for example, in Wolfgang Ertel, “Grundkurs Künstliche Intelligenz: Eine praxisorientierte Einführung.” (Artificial Intelligence Foundation Course: A practical introduction), 3rd edition, Springer Vieweg, Wiesbaden 2013. An AI-based approach for determining observables can be adapted particularly flexibly to data records to be processed and objectives to be pursued. To implement artificial intelligence, a training procedure is usually first applied on the basis of training data in order to train the AI-based structure, for example an artificial neural network, and then apply it to current patient data.

In the method according to one or more example embodiments of the present invention for providing a patient-specific medical action recommendation, determining the one or more observables involves selecting the one or more observables from a database. The database contains a plurality of predetermined observables and/or a plurality of predetermined conditions, each describing one or more feature patterns of medical records of patients and associated with one or more observables. The database also contains a plurality of rules each linking a probability of occurrence of at least one condition to the presence or absence of an observable. Advantageously, statistical empirical values for the presence of medical conditions can be used to estimate their presence or the probability of their presence on the basis of determined observables and to incorporate them in an action recommendation.

The feature pattern can be extracted by a neural network, for example. Particular feature patterns can be associated with different observables. For example, particular image characteristics of the lung can indicate cancer in the liver. In turn, particular observables may be related to this, such as pain, digestive disorders, etc. If the user can confirm this, other action recommendations can follow than if this is not the case. The relationship between a condition and the observables to be queried can be based on specified rules which are automatically taken into account to determine an action recommendation.

In the method according to one or more example embodiments of the present invention for providing a patient-specific medical action recommendation, providing the action recommendation preferably involves determining at least one tentative diagnosis based on the user input, and the action recommendation is preferably established on the basis of the at least one tentative diagnosis. Such a tentative diagnosis can include a probability of occurrence of a disease or medical condition. For example, a determined probability of a condition, such as the occurrence of cancer in the liver, can advantageously be used for automatically making a tentative diagnosis. The user can then confirm or reject the tentative diagnosis based on the information provided to them.

In the method according to one or more example embodiments of the present invention for providing a patient-specific medical action recommendation, the action recommendation preferably includes further examination of the patient using an imaging modality, and the imaging protocol for the imaging modality for examining the patient is determined based on the user input and optionally the other patient data. Advantageously, a next examination step can be prepared on the basis of the automated patient data evaluation process and the dialog process with the user.

In the clinical workflow, further courses of action are often taken by the referring physician who has requested for example a scan or performance of medical imaging using an imaging modality. If the referring physician wants another imaging examination, they will contact the radiologist. The radiologist then sets imaging parameters based on the referring physician's objective. This coordination can also be a multi-step process if, for example, the imaging does not show the desired result. Automated parameter setting can save or at least simplify one step here, because the radiologist only has to agree or can modify the parameters if they deem it necessary. In addition, the coordination between the referring physician and the radiologist can be made more objective, i.e. there is less likelihood of miscommunication between the parties involved.

In the method according to one or more example embodiments of the present invention for providing a patient-specific medical action recommendation, the action recommendation preferably involves recommending that a designated data analysis algorithm be applied to a designated data element from the patient's preferably medical data record, wherein the designated data analysis algorithm is selected from a plurality of available data analysis algorithms and the designated data element is selected from the patient's data record. For example, based on a tentative diagnosis, additional data analysis can be performed and applied selectively to a particular data element. Advantageously, the evaluation of the patient's data record itself can be modified based on an evaluation of the data record in order to iteratively improve the evaluation. The quality of the evaluation can also be influenced by the user, whose specific knowledge of the patient and professional intuition can thus be additionally used to plan the workflow.

In the method according to one or more example embodiments of the present invention for providing a patient-specific medical action recommendation, one or more reference patients from a plurality of comparison patients are preferably identified based on the patient's data record. In addition, comparison information is extracted from the patient data associated with the comparison patients, and the action recommendation is additionally determined based on the comparison information.

In other words, a database containing a plurality of comparison patients is searched for patients similar to a current patient, and these are defined as reference patients for the current patient. If similar patients are identified, comparison information can be extracted from the reference patient's record. This comparison information is data contained in the reference patient's record that is transferable to the current patient based on the similarity of the two patient records.

For example, actions that have been performed for the similar patients or patient records and have possibly proven successful are extracted as comparison information.

The search for similar patients or patient records can be carried out for example by applying an AI-based method designed to extract a feature vector from patient data. The extracted feature vectors can then be compared with one another. The more similar the feature vectors, the more similar the compared patients.

Such a feature vector can include, for example, a so-called image descriptor as well as non-image patient data.

In this variant, searching for similar patients first involves receiving an image descriptor of a patient image of a current patient and retrieving non-image patient data associated with the patient image from the patient's medical record, wherein the current patient image indicates, for example, a medical anomaly. Now, for each of a plurality of candidate images stored in a database, an image descriptor of the candidate image and medical abnormality data indicating a medical abnormality or anomaly known to be indicated by the candidate image is received. Then, for each of the plurality of candidate images, a similarity metric representing a similarity between the image descriptor of the patient image and the image descriptor of the candidate image is determined. In addition, for each of the plurality of candidate images, an initial probability that the medical abnormality indicated by the patient image is the medical abnormality known to be indicated by the candidate image given the non-image patient data associated with the current patient image is determined. For each of the plurality of candidate images, a score is additionally determined based on the determined similarity metric and the determined first probability. Finally, one or more of the candidate images is/are retrieved from the database according to the determined scores.

The first probability can be determined using Bayesian inference, for example.

For each of the plurality of candidate images, the first probability is preferably determined based on a first probability of observing the non-image patient data given a medical abnormality known to be indicated by the candidate image.

This procedure preferably involves determining the first probability from first distribution data representing a first distribution of the medical abnormality known to be indicated by the candidate image among the non-image patient data of a patient population.

For example, the first distribution can be empirically derived.

The non-image patient data can include, for example, demographic data and/or clinical data.

The first probability can be determined based on a second probability of occurrence of the medical abnormality known to be indicated by the candidate image among the medical abnormalities.

This procedure for determining patient similarity preferably involves determining the second probability based on a ratio of the number of candidate images in the database showing the medical abnormality known to be indicated by the candidate image to the total number of candidate images in the database.

The first probability P(D=DI|N) is determined using the following equation:

$$P ( D = {D}_{I} \vert N ) = \frac{P ( N \vert D = {D}_{I} ) P ( D = {D}_{I} )}{P ( N )} , \qquad ( 1 )$$

where P(N|D=DI) is the first probability of observing the non-image patient data N for the medical abnormality DI known to be indicated by the candidate image I, P(D=DI) is the second probability of observing the medical abnormality DI known to be indicated by the candidate image I among the medical abnormalities, and P(N) is the marginal probability of observing the non-image patient data N among other non-image patient data.

The similarity metric is preferably a distance in vector space between the image descriptor of the current patient image and the image descriptor of the candidate image.

The score S is preferably determined using a scoring equation:

S=∥q−i∥²−λlog(P(D=D_{I}|N)), (2)

where q is the image descriptor of the patient image Q, i is the image descriptor of the candidate image I, λ is an adjustable parameter, and P(D=DI|N) is the first probability that the medical anomaly D indicated by the current patient image is the medical anomaly DI known to be indicated by the candidate image I, given the non-image patient data N associated with the current patient image Q.

For illustration, an exemplary calculation of the first probability P(D=DI|N) for three different candidate images I is given. Each candidate image I indicates a different disease D=D1, D2, D3 respectively. In this example, the non-image patient data N of the current patient image Q indicates that the patient is female. The distribution of diseases D1, D2, and D3 by gender FM (FM=female), (M=male) (for example empirically derived) is shown in the following table.

| I | D | FM | M |
|---|---|---|---|
| 1 | D1 | 0.2 | 0.8 |
| 2 | D2 | 0.5 | 0.5 |
| 3 | D3 | 0.8 | 0.2 |

The probability P(N|D=DI) of observing the non-image patient data N (i.e. female), given disease DI known to be indicated by candidate image I, is 0.2 for image 1, 0.5 for image 2, and 0.8 for image 3. In this example, diseases D1, D2, and D3 are assumed to be equally likely to occur in nature, and therefore the second probability P(D=DI) is ⅓ for each of images 1 to 3. In this example, the marginal probability P(N) is 0.5, on the basis that there are equal numbers of male and female patients. Accordingly, using equation (1), the first probability P(D=DI|N) that the medical anomaly D indicated by the current patient image Q is the medical anomaly DI known to be indicated by the candidate image I, given non-image patient data N associated with the current patient image Q, is calculated as follows:

| I | P (D = DI\|N) |
|---|---|
| 1 | 2/15 |
| 2 | 5/15 |
| 3 | 8/15 |

Accordingly, candidate image 3, which indicates the medical anomaly D3 with the highest probability (0.8) of occurrence in females, has the highest ( 8/15) first probability P(D=DI|N). On the other hand, candidate image 1 indicating medical anomaly D1 with the lowest probability (0.2) of occurrence in females has the lowest ( 2/15) first probability P(D=DI|N). Retrieving the candidate images I additionally based on the first probability can therefore help to improve the probability that the medical anomaly indicated in the candidate image I is the same as that of the current patient image, and thus improve the relevance of the retrieved candidate images I to the current patient image Q.

Comparative information can be extracted from the medical record of the reference patients. For example, the medical record of the reference patients can be searched by examinations performed. In addition, diagnostic reports can be searched for usable information using a natural language processing (NLP) algorithm. A description of the use of an NLP algorithm is described, for example, in Prakash M. Nadkarni et al, “Natural language processing: an introduction”, Journal of the American Medical Informatics Association, Volume 18, Issue 5, Sep. 2011, Pages 544-551. Comparison information can include, for example, the diagnostic method used.

The comparison information can optionally be displayed to the user and the user must decide whether to use it in the event.

In the method according to one or more example embodiments of the present invention for providing a patient-specific medical action recommendation, the reference patients are additionally identified based on the user input.

In other words, by taking the user input into account, comparison patients can be searched for in an even more targeted manner. For example, if a user verifies an observable, such as pain in the patient's abdomen, a selective search can be performed for patients who have similar diagnoses. In other words, the observables are included in the feature vector of the patient currently being diagnosed based on the user input.

In the method according to one or more example embodiments of the present invention for providing a patient-specific medical action recommendation, providing the action recommendation preferably involves selecting a designated decision tree from a plurality of predefined decision trees based on the user input and/or the observables and/or the data record. Here, each decision tree has a plurality of different paths and each path provides at least one discrete step for diagnosing/treating the patient. Determining a path within the designated decision tree is based on the user input and/or the observables and/or the data record, and providing the action recommendation is based on the determined path.

The decision trees can be extracted for example from clinical guidelines. Advantageously, the evaluation and decision process for planning a workflow for further treatment or examination steps can be aligned with specified clinical regulations and thus made compatible with the prescribed procedure for treatment and examination planning.

### BRIEF DESCRIPTION OF THE DRAWINGS

The present invention will be explained again in more detail using exemplary embodiments and with reference to the accompanying drawings in which:

FIG. 1 shows a flowchart illustrating a method for providing a patient-specific medical treatment recommendation according to an exemplary embodiment of the present invention,

FIG. 2 shows a block diagram illustrating an action recommendation device according to an exemplary embodiment of the present invention,

FIG. 3 schematically illustrates a clinical examination and treatment system according to an exemplary embodiment of the present invention,

FIG. 4 shows a flowchart illustrating a method for providing a patient-specific medical action recommendation according to a second exemplary embodiment of the present invention,

FIG. 5 shows a flowchart illustrating a method for providing a patient-specific medical action recommendation according to a third exemplary embodiment of the present invention.

### DETAILED DESCRIPTION

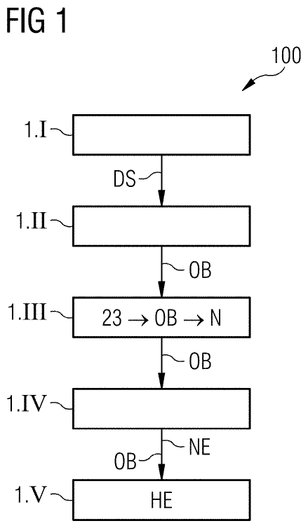

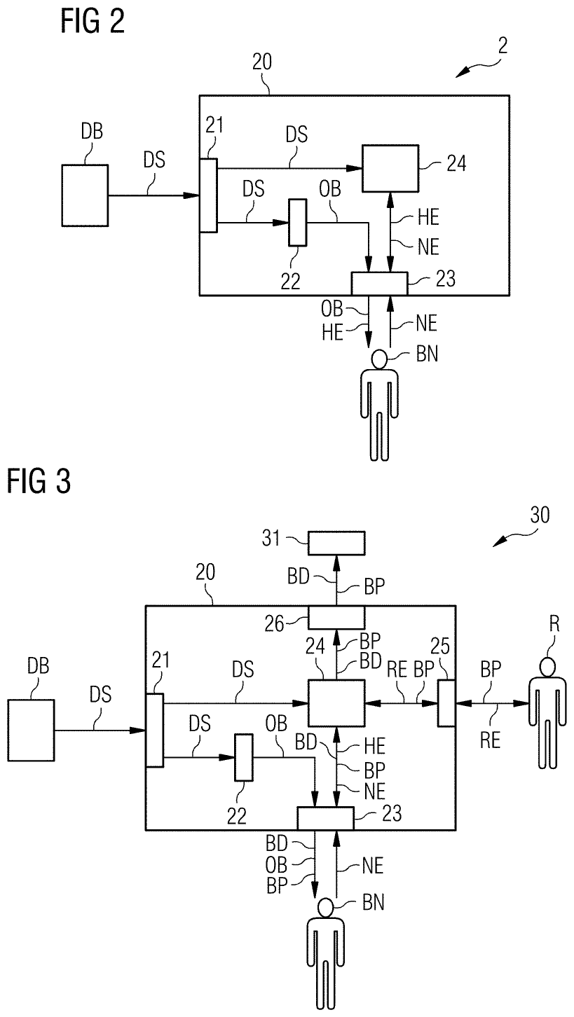

FIG. 1 shows a flowchart 100 illustrating a method for providing a patient-specific medical action recommendation according to a first exemplary embodiment of the present invention. The method can be implemented in a software-based manner on a data processing system, hereinafter also referred to as an action recommendation device 20 (see FIG. 2). In the specific exemplary embodiment, automated treatment planning for an elderly patient suffering from prostate cancer will be illustrated.

In step 1.I, data records DS of the patient, comprising both medical and administrative data records of the patient, are received from one or more databases, for example the PACS and the RIS of a hospital. A medical data record includes, for example, image data of the patient P from previous examinations, annotations by the radiologist, determined symptoms, assessment results, and diagnoses by attending physicians. An administrative data record relates to the administrative recording of the measures and processes already carried out in connection with the patient in question.

In step 1.II, one or more observables OB are now automatically determined based on the data records DS of the patient. The observables OB can be determined by a trained model, also referred to as a state analysis algorithm, which has been made capable of extracting observables OB from the patient-specific data based on training data. As mentioned earlier, observables OB pertain to condition attributes or symptoms of the patient. These condition attributes are extracted from the patient-specific data records DS in an automated manner. For example, condition attributes include information as to whether or not the patient is a smoker. In the context of cancer, the patient's condition may be characterized by the presumption of the existence or non-existence of metastases. Such information can be determined, for example, from image data annotations or explicit findings. Other information can include, for example, laboratory values and their qualitative classification, such as the patient's pKs value or Gleason score.

Condition attributes can also include the existence of a biopsy result and the contents thereof. For example, the biopsy result may address the question as to whether the identified prostate anomaly is a malignant carcinoma.

The patient's condition can also be characterized by information about the patient's current use of medications. For example, the intake of medications and their interactions during examinations may influence the selection and preparation of examinations. Condition attributes may also include a plurality of differential diagnoses.

Symptoms may relate to palpable or measurable phenomena, such as pain in particular areas of the body, its nature, and the frequency or cause of its occurrence or a change in body temperature or similar.

In step 1.III, the automatically determined observables OB describing a clinical condition of the patient are displayed to the user BN, for example a physician, via a user interface 23 (see FIG. 2).

In step 1.IV, the action recommendation device 20 (see FIG. 2) now receives a user input NE from the user BN regarding the displayed observables OB via the user interface 23 (see FIG. 2). This user input NE consists of confirming or rejecting an observable OB or evaluating an observable OB. Thus, additional information is provided by the physician to the action recommendation device 20 and this information is incorporated into the planning of a subsequent workflow for a patient that includes, for example, an examination or treatment.

For example, the user, in this case a physician, may have additional information about observables that is not stored in the PACS/RIS databases containing the patient records DS on the basis of which the observables OB were determined. Thus, by using the user interface 23, a planning process of a workflow can be geared to the physician's current knowledge about a patient. At the same time, the interaction between the action recommendation device 20 and the user BN is machine-prepared as far as possible based on the data records DS available in the PACS/RIS databases, so that the user BN only has to transmit a confirmation message or, if necessary, perform an update of individual observables OB and can simply accept the remaining automatically compiled and presented data.

In step 1.V, automated determination and provision of an action recommendation HE for the user BN regarding further steps for diagnosing/treating the patient takes place based on the user input NE and on the basis of the other determined observables OB of the data records DS. The action recommendation HE can be determined, for example, by applying model-based evaluation schemes to the observables OB determined in the PACS/RIS database as well as the user input NE for a patient.

For example, based on the available patient records DS, the physician BN receives a recommendation to perform further imaging in the chest area of the prostate cancer patient to exclude metastases, or if the occurrence of metastases is already known from the patient records DS or at least is likely, to locate such metastases. Part of the action recommendation HE can be to set the scan parameters BP (see FIG. 3) selected for a planned imaging differently from usual if the patient has particular peculiarities that make it necessary to deviate from the usual approach.

FIG. 2 shows a schematic representation 2 of an action recommendation device 20 according to an exemplary embodiment of the present invention, which device communicates with a database DB and a user BN. The action recommendation device 20 comprises a data receiving interface 21 for receiving a medical data record DS of a patient. The action recommendation device 20 also has a determination unit 22 for automatically determining one or more observables OB based on the data record DS, wherein the one or more observables OB describe a clinical condition of the patient. The action recommendation device 20 also comprises a user interface 23 designed to display the determined observables OB to the user BN and to receive a user input NE from the user BN concerning the displayed observables OB. The user input NE can include, for example, confirmation of the observables OB or modification or rejection of the observables OB. The user input NE is transmitted to a recommendation providing unit 24 which is designed to determine an action recommendation HE for the user BN regarding further steps for diagnosing and/or treating the patient based on the user input NE and the data record DS, and to output the action recommendation HE to the user BN via the user interface 23.

FIG. 3 schematically illustrates a clinical examination and treatment system 30 according to an exemplary embodiment of the present invention. The clinical examination and treatment system 30 comprises an action recommendation device 20 that is expanded compared to the action recommendation device 20 shown in FIG. 2. In addition to communicating with a database DB and a patient's attending physician, also referred to as a user BN, the action recommendation device 20 also communicates with a radiologist R and a medical imaging device

As in the first exemplary embodiment of an action recommendation device 20 shown in FIG. 2, the action recommendation device 20 according to a second exemplary embodiment again comprises a data receiving interface 21 for communicating with a database DB, or rather for receiving a medical data record DS of a patient, a determination unit 22 for determining an observable OB based on the medical record DS, a user interface 23 for communication between the action recommendation device 20 and the user BN, and a recommendation providing unit 24 for determining an action recommendation HE based on a user input NE of the user and on the basis of the determined observable OB. In the second exemplary embodiment shown in FIG. 3, the action recommendation HE includes imaging using a specific imaging protocol BP determined by the action recommendation unit 20 based on the observables OB or the medical data record DS. Now, if the user BN, for example a physician, approves the imaging, the imaging protocol BP associated with this imaging is transmitted to a radiologist R via a radiologist interface 25. The radiologist R assesses the imaging protocol BP, agrees with the suggestion of the action recommendation device 20, or corrects protocol parameters of the imaging protocol BP if necessary. The imaging protocol BP is then received again by the action recommendation device 20 via the aforementioned radiologist interface 25 and transmitted via a device interface 26 to the relevant medical imaging device 31 which acquires a medical image BD of the patient on the basis of the generated imaging protocol BP. The generated medical image BD is then transmitted by the medical imaging device 31 to the action recommendation device 20 which outputs the medical image BD to the user BN via the user interface 23. If the user BN determines that the image BD does not provide the desired result—examination areas where metastases are suspected are not imaged with sufficient contrast, for example—the user BN can either modify the imaging protocol BP on their own initiative or transmit a corresponding message to the radiologist R that the contrast is insufficient. The radiologist R then receives the imaging protocol BP, which may have already been modified, a second time from the action recommendation device 20 and can either confirm or again modify the protocol parameters BP. Subsequently, the modified imaging protocol BP is again sent to the medical imaging device 31 which generates a new image BD of the patient. This process can be repeated a plurality of times until the user BN, i.e. the attending physician, is satisfied with the obtained medical image BD.

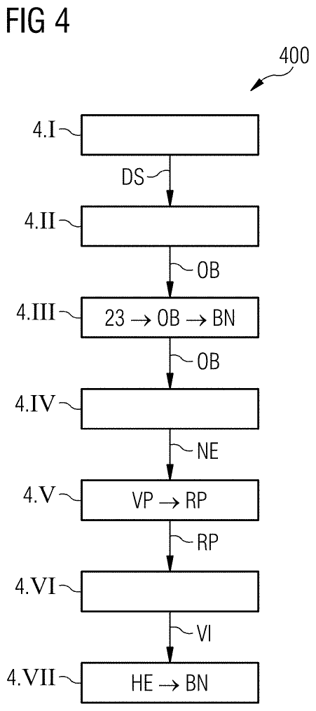

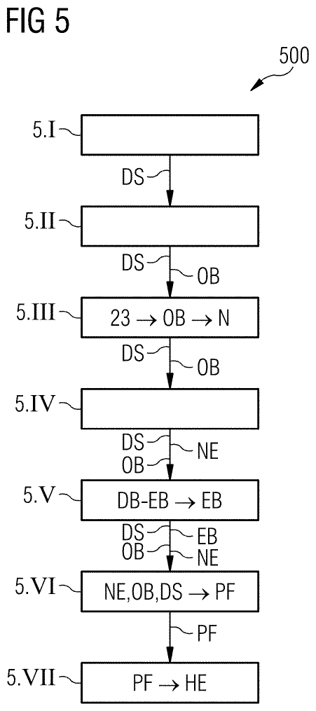

FIG. 4 shows a flowchart 400 illustrating a method for providing a patient-specific medical action recommendation according to a second exemplary embodiment of the present invention. The first four steps 4.I to 4.IV of the method according to a second exemplary embodiment correspond to steps 1.I to 1.IV of the method according to a first exemplary embodiment of the present invention as shown in FIG. 1. These four steps comprise receiving medical records DS of a patient P from a database, automatically determining observables OB based on the patient records DS, displaying the observables OB to the user BN, and a user input NE by the user BN. In addition, in step 4.V, an automated search is now performed in a database containing a plurality of comparison patients VP to find a similar patient that can be used as a reference patient RP for an action recommendation HE. This search is performed based on the data records DS received for the current patient. As explained earlier, such a search can be performed based on an image descriptor of a patient image and non-image patient data of the current patient with the reference patients VP. In step 4.VI, comparison information VI is then extracted from the patient record RP. Such comparison information VI can include, for example, a specific action for examining or treating the reference patient RP, which is output directly in step 4.VII as an action recommendation HE to the user BN. The comparison information VI can also additionally be first processed automatically based on the data DS of the current patient and modified if necessary, and then displayed to the user BN as an action recommendation HE.

FIG. 5 shows a flowchart 500 illustrating a method for providing a patient-specific medical action recommendation according to a third exemplary embodiment of the present invention. The first four steps 5.I to 5.IV again correspond to the first four steps 1.I to 1.IV of the first exemplary embodiment and will not be described again here. In step 5.V, a designated decision tree EB is now selected from a database DB-EB containing a plurality of decision trees EB. Such a decision tree EB comprises rules for examining and treating patients. The selection is based on a user input NE, the determined observables OB and the patient's records DS. Each of the decision trees EB can comprise a plurality of different paths PF and each of these paths PF comprises at least one discrete step for diagnosing or treating a patient. In step 5.VI, a path PF of the designated decision tree EB is now determined based on the user input NE, the observables OB, and the patient's records DS. For example, the attending physician, i.e. the user BN, can provide an input NE based on his knowledge of the patient's situation, leading to the selection of a particular path PF in the designated decision tree EB. In step 5.VII, an action recommendation HE, for example a discrete step to assess or treat the patient, is now determined based on the selected path PF. This action recommendation HE is output to the user BN who can plan the patient's examination or treatment based on the action recommendation HE.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

Finally, it is reiterated that the methods and devices described above are merely preferred exemplary embodiments of the present invention and that the present invention can be varied by persons skilled in the art without departing from the scope of the present invention as set forth in the claims. It is also pointed out for the sake of completeness that the use of the indefinite article “a” or “an” does not exclude the possibility that the features in question may be present more than once. Likewise, the term “unit” does not exclude the possibility that the latter consists of a plurality of components which may also be spatially distributed.

What is claimed is:

1. A computer-implemented method for providing a patient-specific medical action recommendation, the method comprising:
receiving a data record of a patient;
automatically determining one or more observables based on the data record, wherein the one or more observables describe a clinical condition of the patient;
displaying the one or more observables via a user interface;
receiving, via the user interface, user input concerning the one or more observables; and
providing an action recommendation regarding further steps for at least one of diagnosing or treating the patient based on the user input.

2. The computer-implemented method as claimed in claim 1, wherein the automatically determining one or more observables comprises:
applying a state analysis algorithm to the data record, the state analysis algorithm configured to extract observables from data records.

3. The computer-implemented method as claimed in claim 1, wherein the automatically determining one or more observables comprises:
selecting the one or more observables from a database, wherein the database includes
a plurality of observables,
a plurality of conditions, each of the plurality of conditions describing one or more feature patterns of data records of patients, and being associated with one or more of the plurality of observables, and
a plurality of rules, each of the plurality of rules associating a probability of occurrence of at least one condition with a presence or absence of an observable.

4. The computer-implemented method as claimed in claim 1, wherein the providing an action recommendation comprises:
determining at least one tentative diagnosis based on the user input; and
determining the action recommendation based on the at least one tentative diagnosis.

5. The computer-implemented method as claimed in claim 1, wherein
the action recommendation involves further examination of the patient using an imaging modality, and
an imaging protocol for examining the patient using the imaging modality is determined based on the user input.

6. The computer-implemented method according claim 1, wherein
the action recommendation involves applying a designated data analysis algorithm to a designated data element from the data record,
the designated data analysis algorithm is selected from a plurality of available data analysis algorithms, and
the designated data element is selected from the data record.

7. The computer-implemented method as claimed in claim 1, further comprising:
identifying one or more reference patients, from a plurality of comparison patients, based on the data record;
extracting comparison information from patient data associated with the one or more reference patients; and
determining the action recommendation additionally based on the comparison information.

8. The computer-implemented method as claimed in claim 7, wherein the one or more reference patients are additionally identified based on the user input.

9. The computer-implemented method as claimed in claim 1, wherein the providing an action recommendation comprises:
selecting a designated decision tree, from a plurality of decision trees, based on at least one of the user input, the one or more observables or the data record of the patient, wherein each of the plurality of decision trees has a plurality of different paths and each of the plurality of different paths provides at least one discrete step for at least one of diagnosis or treatment of the patient;
determining a path within the designated decision tree based on at least one of the user input, the one or more observables or the data record of the patient; and
providing the action recommendation based on the path within the designated decision tree.

10. An action recommendation device, comprising:
a data receiving interface configured to receive a data record of a patient;
a determination unit configured to automatically determine one or more observables based on the data record of the patient, wherein the one or more observables describe a clinical condition of the patient,
a user interface configured to
display the one or more observables, and
receive user input concerning the one or more observables; and
a recommendation providing unit configured to provide an action recommendation regarding further steps for at least one of diagnosing or treating the patient based on the user input.

11. A clinical examination and treatment system, comprising:
an action recommendation device as claimed in claim 10; and
at least one of a medical examination or treatment device configured to be controlled based on the action recommendation provided by the action recommendation device.

12. A non-transitory computer program product including a computer program loadable into a memory of a computer of a clinical examination and treatment system, the computer program having program sections that, when executed by the computer, cause the clinical examination and treatment system to perform the computer-implemented method of claim 1.

13. A non-transitory computer-readable medium storing program sections that are executable by a computer to cause the computer to perform the computer-implemented method of claim 1.

14. An action recommendation device, comprising:
a memory storing computer executable instructions; and
at least one processor configured to execute the computer executable instructions to cause the action recommendation device to
receive a data record of a patient,
automatically determine one or more observables based on the data record of the patient, wherein the one or more observables describe a clinical condition of the patient,
display the one or more observables,
receive user input concerning the one or more observables, and
provide an action recommendation regarding further steps for at least one of diagnosing or treating the patient based on the user input.

15. The computer-implemented method as claimed in claim 1, wherein the providing provides the action recommendation based on the user input and the data record.

16. The computer-implemented method as claimed in claim 5, wherein the imaging protocol for examining the patient is determined based on the user input and the data record.

17. The action recommendation device of claim 10, wherein the recommendation providing unit is configured to provide the action recommendation based on the user input and the data record.

18. The computer-implemented method as claimed in claim 2, wherein the automatically determining the one or more observables comprises:
selecting the one or more observables from a database, wherein the database includes
a plurality of observables,
a plurality of conditions, each of the plurality of conditions describing one or more feature patterns of data records of patients, and being associated with one or more of the plurality of observables, and
a plurality of rules, each of the plurality of rules associating a probability of occurrence of at least one condition with a presence or absence of an observable.

19. The computer-implemented method as claimed in claim 2, wherein the providing an action recommendation comprises:
determining at least one tentative diagnosis based on the user input; and
determining the action recommendation based on the at least one tentative diagnosis.

20. The computer-implemented method as claimed in claim 2, wherein
the action recommendation involves further examination of the patient using an imaging modality, and
an imaging protocol for examining the patient using the imaging modality is determined based on the user input.
